(19) United States
(12) Patent Application Publication
(10) Pub. No.: US 2023/0088373 A1
(43) Pub. Date: Mar. 23, 2023

(54) PROGRESSIVE INDIVIDUAL ASSESSMENTS USING COLLECTED INPUTS

(71) Applicant: Yamaha Motor Corporation, USA, Cypress, CA (US)

(72) Inventor: Dave PARK, Kennesaw, GA (US)

(21) Appl. No.: 18/052,883

(22) Filed: Nov. 4, 2022

Related U.S. Application Data
(63) Continuation of application No. PCT/US2021/031147, filed on May 6, 2021.
(60) Provisional application No. 63/022,154, filed on May 8, 2020.

(51) Int. Cl. A61B 5/16; G06N 20/00

(52) U.S. Cl. CPC A61B 5/165; G06N 20/00

(57) ABSTRACT

One or more aspects of the present application relate to systems and methods for collecting, processing and generating information regarding individual users. Individual users can generate information that are associated with, or otherwise directed to, cognitive, emotional, physical or social interactions. Aspects of such information may be considered active or passive in nature. For example, some embodiments of the collected information are associated with individuals or groups of individuals providing information, such as by interacting with textual or graphical user interfaces. Other embodiments of the collected information are associated with services or devices associated with the individual users that collect or generate information associated with user behavior or interaction. Cumulatively, a collection of passive and active information associated with an individual, or attributed to an individual, allow an individual information management system to generate a set of assessments, such as cognitive, emotional, physical or social assessments for the individual.

### PRIORITY CLAIM

The present application is a continuation of PCT/US2021/031147, filed May 6, 2021 which claims the benefit of U.S. Provisional Application No. 63/022,154, filed May 8, 2020.

### BACKGROUND

Generally described, computing devices and communication networks can be utilized to exchange data and/or information. In a common application, a computing device can request content from another computing device via the communication network. For example, a user at a personal computing device can utilize a browser application to request a content page (e.g., a network page, a Web page, etc.) from a server computing device via the network (e.g., the Internet). In such embodiments, the user computing device can be referred to as a client computing device and the server computing device can be referred to as a content provider. In another embodiment, the user computing device can collect or generate information and provide the collected information to a server computing device for further processing or analysis.

In the context of individual users, a service provider can collect information provided by computing devices associated with individual users. The collected information can include information directly provided by the individual users, such as via textual or graphical input interfaces. The collected information can also include information related to the individual user that is measured or generated by a computing device. Service providers can then use the collected information to make assessments about individual users.

### BRIEF DESCRIPTION OF THE DRAWINGS

Throughout the drawings, reference numbers may be re-used to indicate correspondence between referenced elements. The drawings are provided to illustrate example embodiments described herein and are not intended to limit the scope of the disclosure.

FIG. 1 is a block diagram of a network environment that includes one or more devices associated with individual users, one or more devices associated with third party information providers, and an individual information management service for processing user information according to one embodiment;

FIG. 2A is a block diagram of illustrative components of a user computing device configured to access or provide individual content in accordance with an illustrative embodiment;

FIG. 2B is a block diagram of illustrative components of a user computing device configured to access or provide individual content in accordance with an alternative embodiment;

FIG. 3A is a block diagram of illustrative components of a cognitive assessment component device configured to process individual user information to generate cognitive assessments in accordance with an illustrative embodiment;

FIG. 3B is a block diagram of illustrative components of an emotional assessment component device configured to process individual user information to generate emotional assessments in accordance with an illustrative embodiment;

FIG. 3C is a block diagram of illustrative components of a physical assessment component device configured to process individual user information to generate physical assessments in accordance with an illustrative embodiment;

FIG. 3D is a block diagram of illustrative components of a social assessment component device configured to process individual user information to generate social assessments in accordance with an illustrative embodiment;

FIG. 4 is a block diagram of illustrative components of an individual information management component device configured to process individual user information, manage the generation of various assessments based on inputted information and execute responsive actions in accordance with an illustrative embodiment;

FIGS. 5A-5B are block diagrams illustrative of the environment of FIG. 1 illustrating the interaction between user computing devices and the individual information management service to make cognitive, emotional, physical and social assessments in accordance with an illustrative embodiment;

FIG. 6A is a flow diagram illustrative of a user information processing routine implemented by an individual information management service in accordance with an aspect of the present application;

FIG. 6B is a flow diagram illustrative of an assessment processing routine implemented by an individual information management service or assessment controller in accordance with an aspect of the present application;

FIG. 7A is a flow diagram illustrative of a cognitive assessment processing-sub-routine implemented by an individual information management service in accordance with an aspect of the present application;

FIG. 7B is a flow diagram illustrative of an emotional assessment processing-sub-routine implemented by an individual information management service in accordance with an aspect of the present application;

FIG. 7C is a flow diagram illustrative of a physical assessment processing-sub-routine implemented by an individual information management service in accordance with an aspect of the present application; and

FIG. 7D is a flow diagram illustrative of a social assessment processing-sub-routine implemented by an individual information management service in accordance with an aspect of the present application.

### DETAILED DESCRIPTION

One or more aspects of the present application relate to systems and methods for collecting, processing and generating information regarding individual users. Individual users can generate information that are associated with, or otherwise directed to, cognitive, emotional, physical or social interactions. Aspects of such information may be considered active or passive in nature. For example, some embodiments of the collected information are associated with individuals or groups of individuals providing information, such as by interacting with textual or graphical user interfaces. Other embodiments of the collected information are associated with services or devices associated with the individual users that collect or generate information associated with user behavior or interaction. Cumulatively, a collection of passive and active information associated with an individual, or attributed to an individual, allow an individual information management system to generate a set of assessments, such as cognitive, emotional, physical or social assessments for the individual.

By way of illustration, cognitive assessments based on passive user information, active information, or a combination thereof, can generally correspond to a measure of mental functions, such as memory, language, and the ability to recognize objects. By way of illustrative example, cognitive assessments can correspond to a determination or characterization of confusion, attention, decision-making, visual special problems and memory loss. Emotional assessment based on passive user information, active information, or a combination thereof, can generally correspond to a measure of emotional dynamics related to individual growth and difficulties that may be present, also referred to as a personality assessment. By way of illustrative example, emotional assessment can correspond to a determination or characterization of depression, apathy, personality changes, mood swings or variations and agitation. Physical assessment based on passive user information, active information, or a combination thereof, can generally corresponds to a measure of anatomical state or performance of an individual. By way of illustrative example, physical assessment can correspond to a determination or characterization of visual hallucinations, auditory hallucinations, olfactory hallucinations, tactile hallucinations, eye movement, balance, falls, facial expression, reduced strength, reflexes, muscle stiffness, muscle contracture, jerking, tremors, gait, REM sleep, drowsiness, staring, disorganized speech, incoordination, posture, mispronunciation, non-verbal gesturing and hearing/visual skills. Finally, social assessment based on passive user information, active information, or a combination thereof, can generally correspond to a measure of social dynamics and difficulties that may be present. By way of illustrative example, social assessments can correspond to a determination of depression, reduced social interaction, withdrawn and sedentariness.

Traditional approaches to processing individual information based on cognitive, emotional, physical or social interactions can include a service provider one or more inputs to make one or more assessments. Such assessments, however, can be more limited terms of applying traditional forms of inputs such as user provided answers to questions or processing actions/answers to specific tests. More specifically, traditional approaches do not consider combinations of active inputs and passive inputs for conducting individual assessments. Additionally, traditional approaches do not facilitate comparative analysis of assessments based on historical information that can be indicative of decay/erosion of cognitive, emotional, physical or social behavior for identified individuals. Such lack of utilization of historical information does not allow for individualized assessments and implementation of a comparative analysis based on dynamic factors, such as individual profiles, age, and the like.

Based on at least a portion of the above identified inefficiencies and deficiencies, one or more aspects of the present application include the utilization of a set of individual information collected from various devices or other sources to form passive and active inputs associated with one or more individuals. Illustratively, passive inputs can correspond to information associated with or derived from an individual's interaction in an environment such as utilization of mobile devices, interaction with computing devices, interaction with vehicles, use of social media, and the like. Illustratively, active inputs can correspond to information associated with measurements of an individual's physical movement, such as use of exercise equipment, measurement of health inputs or medical conditions, medical history, user inputs (e.g., “how are you feeling?”), and the like. The set of individual inputs may be provided from different devices associated, or attributed with an individual, such as a mobile device, one or more vehicles, customized appliance and other computing devices. Additionally, the set of individual inputs may also come from third-party services, such as social media network services, calendaring services, medical records/profile services, and the like.

In accordance with aspects of the present application, an individual information management service can collect the set of inputs and process the set of inputs in order to conduct a plurality of assessments, including cognitive assessments, emotional assessments, physical assessments and social assessments. The individual information management service can utilize one or more assessment services that can execute machine-learning algorithms to process the set of collected inputs and additional information to make the plurality of individual assessments. For example, the individual information management service can configure individual components or services to make assessments based on trained learning models for each respective type of assessment. The plurality of assessments may be stored or incorporated into historical information.

The individual information management service can further implement a comparative engine or process to compare a current plurality of assessment to historical assessments. In this regard, the individual information management service can compare potential differences or deviations against thresholds. Individual thresholds may be dynamic in nature and may be set or modified based on age-based criteria, individual profiles, regional or cultural criteria, and the like. For example, the individual information management service can apply different threshold based on age ranges so that assessments may change as individual's age or between different individuals associated with different age ranges. Additionally, the individual information management service can apply different types of thresholds based on the type of assessment (e.g., cognitive, emotional, physical, social or diet). Still further, the individual information management service can further include some dynamic thresholds that can be adjusted based on manual inputs (e.g., health care provider), or based on historical measurable differences for individuals, such as for threshold information or meta-data maintained in individual profile information.

Illustratively, the individual information management service can utilize the comparative differences and threshold comparison to determine one or more responsive actions, that can be considered to be corrective actions or mitigation techniques and that can be provided or recommended for the individual. The responsive actions will be generally described as “corrective actions,” but such actions should not be interpreted as requiring some form of medical diagnosis and medically approved course of treatment. The corrective actions can be automatically initiated and configured based on the results of the comparative assessments, such as selecting which corrective actions or settings/values for the corrective actions based on the comparative assessments. The corrective actions may be based, at least in part, on an individual plan that can include customized or specified corrective actions based on individual preferences, medical staff input, and the like. The individual information management service can further record and measure individual responses to the corrective actions as further input to historical information. For example, an individual's response to corrective action can then be provided as confirmation regarding an assessment, in selecting the corrective actions or configuring/setting values for determined corrective actions.

By utilizing separate assessments, historical assessment data and thresholding, one or more aspects of the present application achieve various benefits over traditional assessment approaches. For example, aspects of the present application can facilitate comparative assessments and thresholding to identify early signs of diseases, such as dementia, that would not be generally cognizant to a health care provider via a single assessment or examination. Additionally, the set of active and passive inputs utilized by the individual information management service can represent data that would not typically be available to individual cognitive, emotional, physical, social or diet assessments. Additionally, aspects of the present application can utilize machine learning or other optimizes to facilitate large-scale data processing and relationship determination that are outside of the scope of traditional data processing approaches, including the potential intra-relationships/influences of cognitive, emotional, physical, social and diet assessments. Other benefits or distinguishing factors may be also be realized by one or more aspects of the present application.

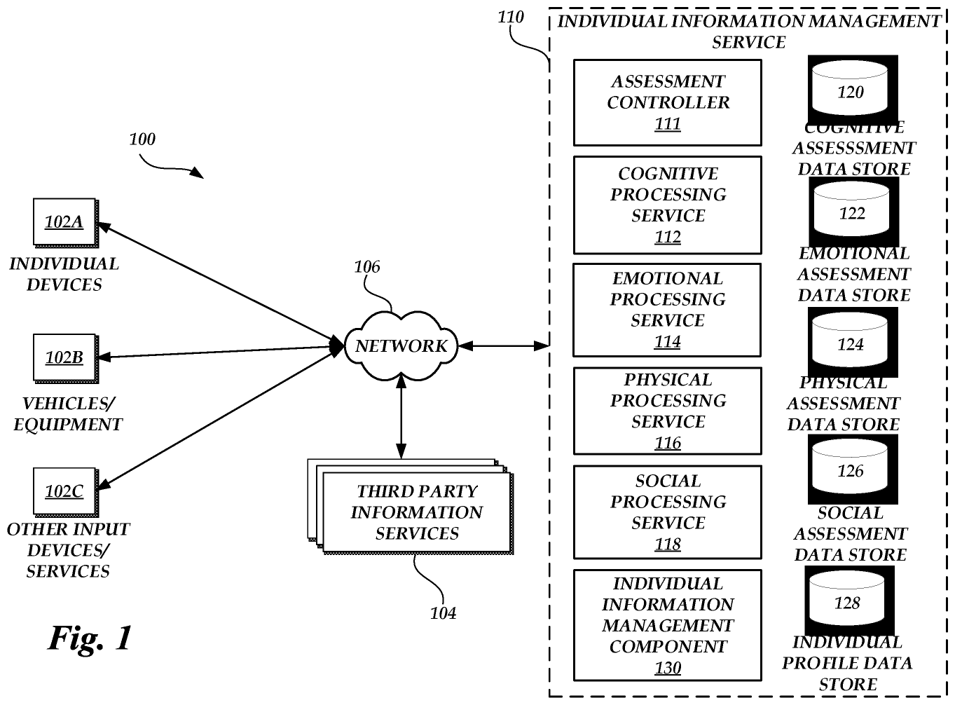

FIG. 1 is a block diagram of a network environment 100 that includes one or more devices associated with individuals 102, one or more devices associated with third-party information providers 104, and an individual information management service 110 according to one embodiment. The environment 100 includes a plurality of devices 102 utilized by individuals that either are specifically configured to elicit information utilized by the individual information management service 110 or that relate to collected information that is processed by the individual information management service 110. By way of illustration, the plurality of devices 102 can include individual devices 102A accessible by individuals and that generate individual data, vehicles/equipment 102B that may be used by individuals, exclusively or non-exclusively, and other input sources/services 102C that generate data attributable to individuals. One skilled in the relevant art will appreciate that the illustration of different individual devices 102A, 102B, and 102C is only illustrative in nature and is not intended to limit the types of devices that can generate individual data or require a categorization of individual devices.

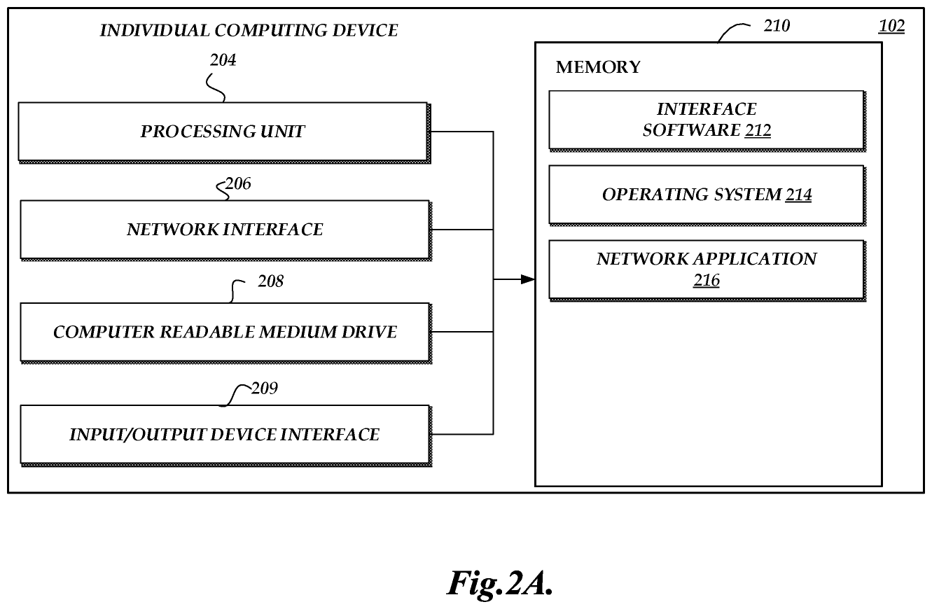

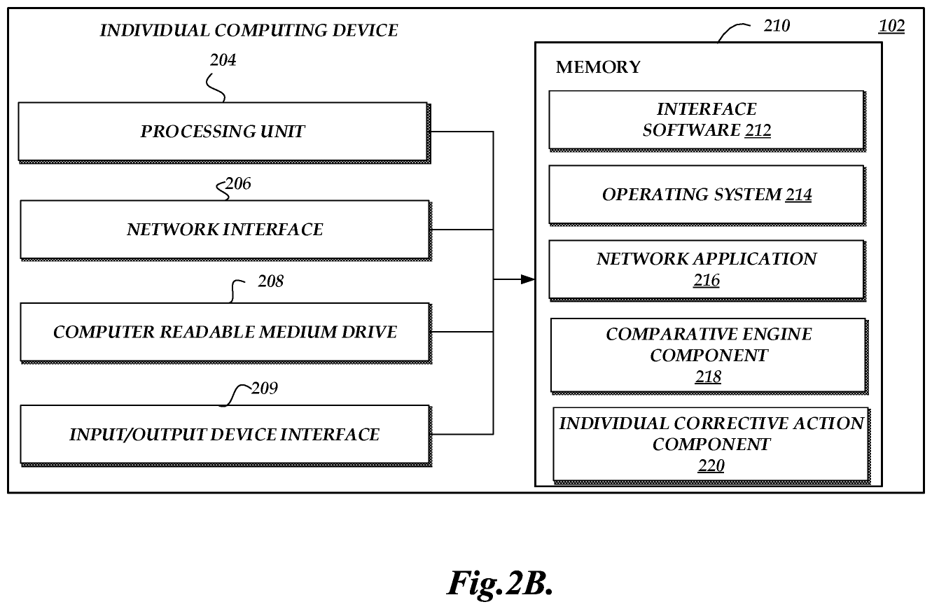

Illustratively, individual accessing computing devices 102 may correspond to a laptop or tablet computer, personal computer, wearable computer, server, personal digital assistant (PDA), hybrid PDA/mobile phone, mobile phone, electronic book reader, set-top box, camera, appliance (e.g. a thermostat or refrigerator), controller, digital media player, watch, glasses, a home or car device, Internet of Thing (“IoT”) devices, virtual reality or augmented reality devices, wearable accessories, and the like. Still further, the individual computing devices 102 can represent devices or components that have alternative purposes, but have been configured to function as an individual device as described herein. For example, the individual device 102 can correspond to a musical instrument that includes some form of computing capability or sensors to provide information associated with a user's manipulation of the instrument. In another example, a bicycle, such as an electronic bicycle, can be outfitted with sensors to capture different types of user input or interaction or use thereof. In yet another example, a mobile device may be configured to provide or identify audible levels indicative of speech levels of the user according to a predefined passage (e.g., a comparative text passage) or according to individual usage of the mobile device over time. Each computer device 102 may optionally include one or more data stores (not shown in FIG. 1) including various applications or computer-executable instructions, such as web browsers or media player software applications, used to implement the embodiments disclosed herein. Illustrative components of an individual device 102 (e.g., 102A, 102B or 102C) will be described with regard to FIG. 2. Still further, although not illustrated in FIG. 1, in some embodiments, different types of individual devices 102 may be applicable to different individuals or different sets of individual devices may change over time based on the type of activities or interaction experienced by the individuals. For example, an individual may access different types of vehicles that can generate individual information depending on the age, fitness level, financial means, etc. that can vary over time.

The environment 100 includes a plurality of devices 104 or network of devices utilized by third party information providers, generally referred to as third party information services 104 to submit information. Third-party information sources 104 may include any number of different computing devices capable of communicating with the network 106, via a direct connection or via an intermediary. For example, individual third-party information sources may correspond to a laptop or tablet computer, personal computer, wearable computer, server, personal digital assistant (PDA), hybrid PDA/mobile phone, mobile phone, electronic book reader, set-top box, camera, appliance (e.g., a thermostat or refrigerator), controller, digital media player, watch, glasses, a home or car device, Internet of Thing (“IoT”) devices, virtual reality or augmented reality devices, and the like. Each third-party information sources 104 may optionally include one or more data stores (not shown in FIG. 1) including various applications or computer-executable instructions, such as web browsers or media player software applications, used to implement the embodiments disclosed herein.

As will be explained in greater detail below, the applications can be configured to provide information regarding active inputs or passive inputs related to an individual, such as social media usage, network interaction, speech patterns, word usage, device usage, location tracking, medical histories, medical suggestions, and the like. For example, the third-party information sources 104 can provide individual information related to utilization or metrics associated with operation of mobile device (e.g., speech pattern information, call frequency, data usage, etc.). In another example, the third-party information sources 104 can include social media information related to interaction with social media resources, such as frequency of access or sharing content, analysis of shared content, metrics regarding social media relationships, and the like. In still another example, the third-party information sources 104 can include medical services that can include medical history information or recommendations/suggestions regarding corrective actions. In yet another example, the third-party information sources 104 can provide information related to specific interactions between a user and other identified individuals, such as calendaring information (e.g., lunch with X) or category/types of interaction. Other examples of third-party information sources 104 are considered to be within the scope of the present application.

Network 106 may be any wired network, wireless network, or combination thereof. In addition, the network 106 may be a personal area network, local area network, wide area network, cable network, fiber network, satellite network, cellular telephone network, data network, or combination thereof. In the example environment of FIG. 1, network 106 is a global area network (GAN), such as the Internet. Protocols and components for communicating via the other aforementioned types of communication networks are well known to those skilled in the art of computer communications and thus, need not be described in more detail herein. While each of the individual computing devices 102A, 102B, and 102C, the third-party information sources 104, and the individual information management service 110 are depicted as having a single connection to the network 106, individual components of the individual computing devices 102A, 102B, and 102C, the third-party information sources 104, and the individual information management service 110 may be connected to the network 106 at disparate points. Accordingly, communication times and capabilities may vary between the components of FIG. 1. Likewise, although FIG. 1 is illustrated as having a single network 106, one skilled in the relevant art will appreciate that the environment 100 may utilize any number or combination of networks.

In accordance with embodiments, the individual information management service 110 includes one or more servers for receiving content from the individual computing devices 102A, 102B, and 102C and the third-party information sources 104 for processing the content to conduct one or more assessments and identify possible corrective actions as described herein. As described in further detail below, the individual information management service 110 includes a cognitive processing service 112, an emotional processing component 114, a physical processing service 116, a social processing service 118, and a diet processing service 119 that can be configured to generate respective cognitive, emotional, physical and social assessments as described herein. The individual information management service 110 can further include an assessment controller 111 that can be configured to initiate and configure individual assessments from the cognitive processing service 112, emotional processing component 114, physical processing service 116,social processing service 118, and a diet processing service 119.

The individual information management service 110 can further include an individual information management component 130 can implement comparative analysis of individual assessments and identify corrective actions as described herein. Although the various services 112-119 and 130 associated with the individual information management service 110 are illustrated as single components, each individual service 112-119 and 130 may be implemented in a number of different instantiated components, including virtualized resources. For example, the individual information management component 130 may correspond to a plurality of devices or virtual machine instances that are configured to implement different types of comparative assessments. Still further, as will be described in FIG. 2B, in one embodiment, at least some portion of the functionality of the individual information management component 130 may be implemented in an individual user device 102A, such as a mobile device that includes a customized application or set of applications or standard household appliances that are configured with customized applications, such as an interaction capture application. In such embodiments, the individual information management component 130 may be omitted altogether or the individual information management component 130 may work in conjunction with any applications on the individual device 102.

The individual information management service 110 further can include a number of data stores for maintaining different information. The data stores include cognitive assessment data store 120, an emotional assessment data store 122, a physical assessment data store 124, a social assessment data store 126, and a diet assessment store 127. The data stores can further include a user profile data store 128 that can maintain individual profile information, such as configurations for the assessments, social media contacts, medical history information, calendaring information, other historical information, customized corrective actions and any additional information related to individuals that may be utilized in various processes defined herein. Although illustrated as individual data stores, the data stores 120, 122, 124, 126, 127 and 128 can correspond to multiple data stores, distributed data stores, or variations thereof

It will be appreciated by those skilled in the art that the environment 100 may have fewer or greater components than are illustrated in FIG. 1. Thus, the depiction of the environment 100 in FIG. 1 should be taken as illustrative. For example, in some embodiments, components of the individual information management service 110 may be executed by one more virtual machines implemented in a hosted computing environment. A hosted computing environment may include one or more rapidly provisioned and released computing resources, which computing resources may include computing, networking or storage devices. Additionally, while such components are illustrated as logically being logically grouped in FIG. 1, one skilled in the relevant art will appreciate that one or more aspects of the present application can include the individual information management service 110 as being implemented in multiple geographic areas. Additionally, not all geographic areas hosting portions of the individual information management service 110 will necessarily have all the same components or combination of components.

FIG. 2A depicts one embodiment of an architecture of an illustrative individual computing device 102, such as a personal computer, tablet computer, smartphone, or other device, that can generate content and process content requests in accordance with the present application. FIG. 2A is illustrative of the general framework of an individual computing device regardless of the different characterizations of the individual devices 102A, 102B, and 102C (FIG. 1). The general architecture of the client device 102 depicted in FIG. 2A includes an arrangement of computer hardware and software components that may be used to implement aspects of the present disclosure. As illustrated, the client device 102 includes a processing unit 204, a network interface 206, a computer readable medium drive 208, an input/output device interface 209, an optional display 202, and an input device 224, all of which may communicate with one another by way of a communication bus. In various embodiments, components such as the display 202 and/or the input device 224 may be integrated into the client device 102, or they may be external components that are coupled to the device 102.

The network interface 206 may provide connectivity to one or more networks or computing systems, such as the network 106 of FIG. 1. The processing unit 204 may thus receive information and instructions from other computing systems or services via a network. The processing unit 204 may also communicate to and from memory 210 and further provide output information for an optional display 202 via the input/output device interface 220. The input/output device interface 209 may also accept input from the optional input device 224, such as a keyboard, mouse, digital pen, etc. In some embodiments, the client device 102 may include more (or fewer) components than those shown in FIG. 2A.

The memory 210 may include computer program instructions that the processing unit 204 executes in order to implement one or more embodiments. The memory 210 generally includes RAM, ROM, or other persistent or non-transitory memory. The memory 210 may store an operating system 214 that provides computer program instructions for use by the processing unit 204 in the general administration and operation of the client device 102. The memory 210 may further include computer program instructions and other information for implementing aspects of the present disclosure. For example, in one embodiment, the memory 210 includes a network application 216, such as browser application, for accessing and generating individual information.

FIG. 2B depicts another embodiment of an architecture of an illustrative individual computing device 102, such as a personal computer, tablet computer, smartphone, or other device, that can generate content and process content requests in accordance with the present application. FIG. 2B is illustrative of the general framework of an individual computing device regardless of the different characterizations of the individual devices 102A, 102B, and 102C (FIG. 1). FIG. 2B references the same numerals described above with regard to FIG. 2A for a user device 102.

As described above, in some embodiments, one or more aspects of the functionality associated with the individual information management component 130 may be implemented in an individual user device 102. Accordingly, with reference to FIG. 2B, the memory 210 includes a comparative engine component 218 for generating comparative assessments of assessments described herein. The comparative engine component 218 may illustratively correspond to machine learning algorithms to obtain inputs, process the inputs and generate characterizations of the presence of or value of physical indicators. The memory 210 can also include an individual corrective action component 220 for determining and implementing corrective actions for an individual based on the results of the comparative assessments, including customized assessments based on user profile information.

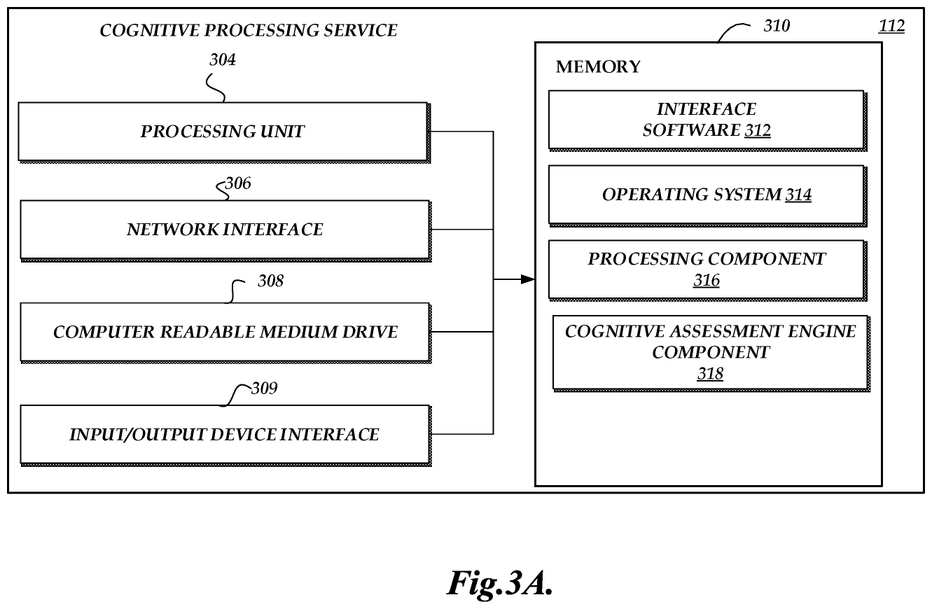

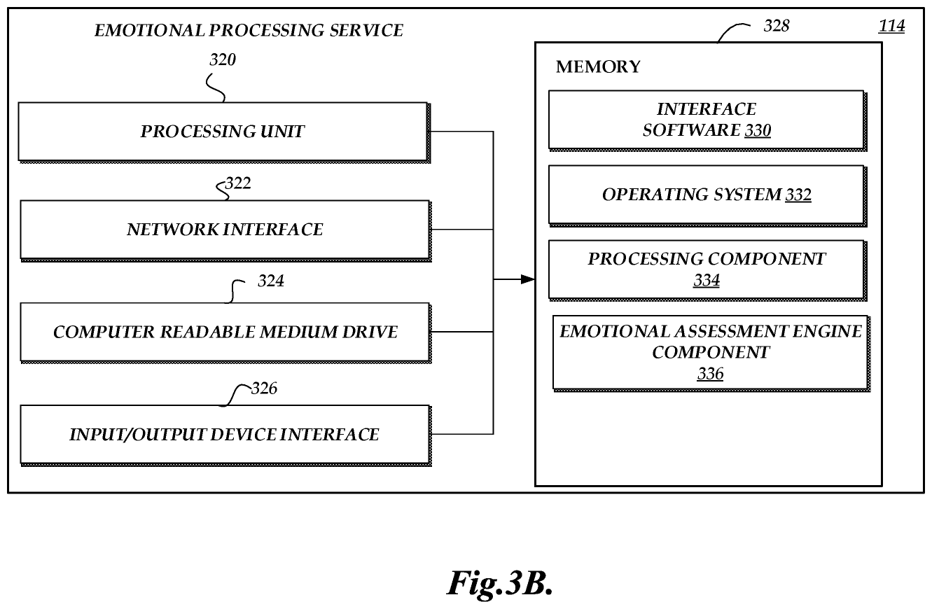

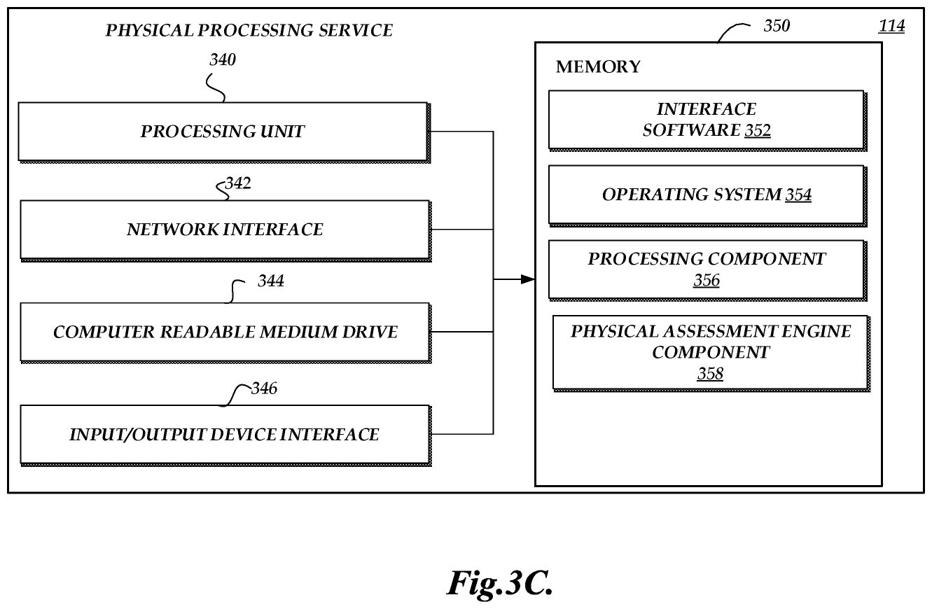

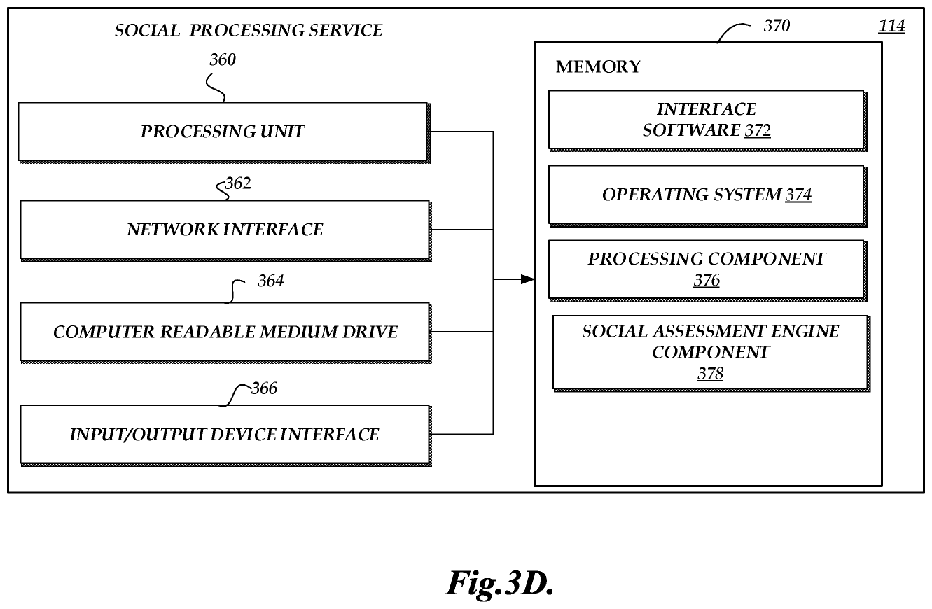

FIG. 3A depicts one embodiment of an architecture of an illustrative server for implementing the cognitive processing service/component 112 as described. The general architecture of the cognitive processing service/component 112 depicted in FIG. 3A includes an arrangement of computer hardware and software components that may be used to implement aspects of the present disclosure. As illustrated, the cognitive processing service/component 112 includes a processing unit 304, a network interface 306, a computer readable medium drive 308, and an input/output device interface 309, all of which may communicate with one another by way of a communication bus. The components of the cognitive processing service/component 112 may be physical hardware components or implemented in a virtualized environment.

The network interface 306 may provide connectivity to one or more networks or computing systems, such as the network 106 of FIG. 1. The processing unit 304 may thus receive information and instructions from other computing systems or services via a network. The processing unit 304 may also communicate to and from memory 310 and further provide output information for an optional display via the input/output device interface 306. In some embodiments, the cognitive processing service/component 112 may include more (or fewer) components than those shown in FIG. 3A.

The memory 310 may include computer program instructions that the processing unit 304 executes in order to implement one or more embodiments. The memory 310 generally includes RAM, ROM, or other persistent or non-transitory memory. The memory 310 may store an operating system 314 that provides computer program instructions for use by the processing unit 306 in the general administration and operation of the cognitive processing service/component 112. The memory 310 may further include computer program instructions and other information for implementing aspects of the present disclosure. For example, in one embodiment, the memory 310 includes processing software 316 for receiving and processing inputs utilized to make cognitive assessments of individuals. Additionally, the memory 310 includes a cognitive assessment engine component 318 that is configured to process the inputs and generate cognitive assessments. The cognitive assessment engine component 318 may illustratively correspond to machine learning algorithms to obtain inputs, process the inputs and generate characterizations of the presence of or value of cognitive indicators. Examples of such cognitive assessments can include, but is not limited to a determination or characterization of confusion, attention, decision-making, visual special problems and memory loss.

FIG. 3B depicts one embodiment of an architecture of an illustrative server for implementing the emotional processing service/component 114 as described. The general architecture of the emotional processing service/component 114 depicted in FIG. 3B includes an arrangement of computer hardware and software components that may be used to implement aspects of the present disclosure. As illustrated, the emotional processing service/component 114 includes a processing unit 320, a network interface 322, a computer readable medium drive 324, and an input/output device interface 326, all of which may communicate with one another by way of a communication bus. The components of the emotional processing service/component 114 may be physical hardware components or implemented in a virtualized environment.

The network interface 322 may provide connectivity to one or more networks or computing systems, such as the network 106 of FIG. 1. The processing unit 320 may thus receive information and instructions from other computing systems or services via a network. The processing unit 320 may also communicate to and from memory 328 and further provide output information for an optional display via the input/output device interface 326. In some embodiments, the emotional processing service/component 114 may include more (or fewer) components than those shown in FIG. 3B.

The memory 328 may include computer program instructions that the processing unit 320 executes in order to implement one or more embodiments. The memory 328 generally includes RAM, ROM, or other persistent or non-transitory memory. The memory 328 may store an operating system 322 that provides computer program instructions for use by the processing unit 320 in the general administration and operation of the emotional processing service/component 114. The memory 328 may further include computer program instructions and other information for implementing aspects of the present disclosure. For example, in one embodiment, the memory 328 includes processing software 334 for receiving and processing inputs utilized to make emotional assessments of individuals. Additionally, the memory 328 includes an emotional assessment engine component 336 that is configured to process the inputs and generate emotional assessments. The emotional assessment engine component 336 may illustratively correspond to machine learning algorithms to obtain inputs, process the inputs and generate characterizations of the presence of or value of emotional indicators. Examples of such emotional assessments includes, but is not limited to, a determination or characterization of depression, apathy, personality changes, mood swings or variations and agitation.

FIG. 3C depicts one embodiment of an architecture of an illustrative server for implementing the physical processing service/component 116 as described. The general architecture of the physical processing service/component 116 depicted in FIG. 3C includes an arrangement of computer hardware and software components that may be used to implement aspects of the present disclosure. As illustrated, the physical processing service/component 116 includes a processing unit 340, a network interface 342, a computer readable medium drive 344, and an input/output device interface 346, all of which may communicate with one another by way of a communication bus. The components of the physical processing service/component 116 may be physical hardware components or implemented in a virtualized environment.

The network interface 342 may provide connectivity to one or more networks or computing systems, such as the network 106 of FIG. 1. The processing unit 340 may thus receive information and instructions from other computing systems or services via a network. The processing unit 342 may also communicate to and from memory 350 and further provide output information for an optional display via the input/output device interface 346. In some embodiments, the physical processing service/component 116 may include more (or fewer) components than those shown in FIG. 3C.

The memory 350 may include computer program instructions that the processing unit 340 executes in order to implement one or more embodiments. The memory 350 generally includes RAM, ROM, or other persistent or non-transitory memory. The memory 350 may store an operating system 354 that provides computer program instructions for use by the processing unit 340 in the general administration and operation of the physical processing service/component 116. The memory 350 may further include computer program instructions and other information for implementing aspects of the present disclosure. For example, in one embodiment, the memory 350 includes processing software 356 for receiving and processing inputs utilized to make physical assessments of individuals. Additionally, the memory 350 includes a physical assessment engine component 358 that is configured to process the inputs and generate physical assessments. The physical assessment engine component 358 may illustratively correspond to machine learning algorithms to obtain inputs, process the inputs and generate characterizations of the presence of or value of physical indicators. Examples of such physical assessments can include, but are not limited to, a determination or characterization of visual hallucinations, auditory hallucinations, olfactory hallucinations, tactile hallucinations, eye movement, balance, falls, facial expression, reduced strength, reflexes, muscle stiffness, muscle contracture, jerking, tremors, gait, REM sleep, drowsiness, staring, disorganized speech, incoordination, posture, mispronunciation, non-verbal gesturing and hearing/visual skills.

FIG. 3D depicts one embodiment of an architecture of an illustrative server for implementing the social processing service/component 118 as described. The general architecture of the social processing service/component 118 depicted in FIG. 3D includes an arrangement of computer hardware and software components that may be used to implement aspects of the present disclosure. As illustrated, the social processing service/component 118 includes a processing unit 360, a network interface 362, a computer readable medium drive 364, and an input/output device interface 366, all of which may communicate with one another by way of a communication bus. The components of the social processing service/component 118 may be physical hardware components or implemented in a virtualized environment.

The network interface 362 may provide connectivity to one or more networks or computing systems, such as the network 106 of FIG. 1. The processing unit 360 may thus receive information and instructions from other computing systems or services via a network. The processing unit 362 may also communicate to and from memory 370 and further provide output information for an optional display via the input/output device interface 366. In some embodiments, the social processing service/component 118 may include more (or fewer) components than those shown in FIG. 3D.

The memory 370 may include computer program instructions that the processing unit 360 executes in order to implement one or more embodiments. The memory 370 generally includes RAM, ROM, or other persistent or non-transitory memory. The memory 370 may store an operating system 374 that provides computer program instructions for use by the processing unit 360 in the general administration and operation of the social processing service/component 118. The memory 370 may further include computer program instructions and other information for implementing aspects of the present disclosure. For example, in one embodiment, the memory 370 includes processing software 376 for receiving and processing inputs utilized to make social assessments of individuals. Additionally, the memory 370 includes a social assessment engine component 378 that is configured to process the inputs and generate social assessments. The social assessment engine component 378 may illustratively correspond to machine learning algorithms to obtain inputs, process the inputs and generate characterizations of the presence of or value of physical indicators. Examples of such social assessments can include, but are not limited to, a determination of depression, reduced social interaction, withdrawn and sedentariness, confusion, attention, decision-making, visual special problems and memory loss.

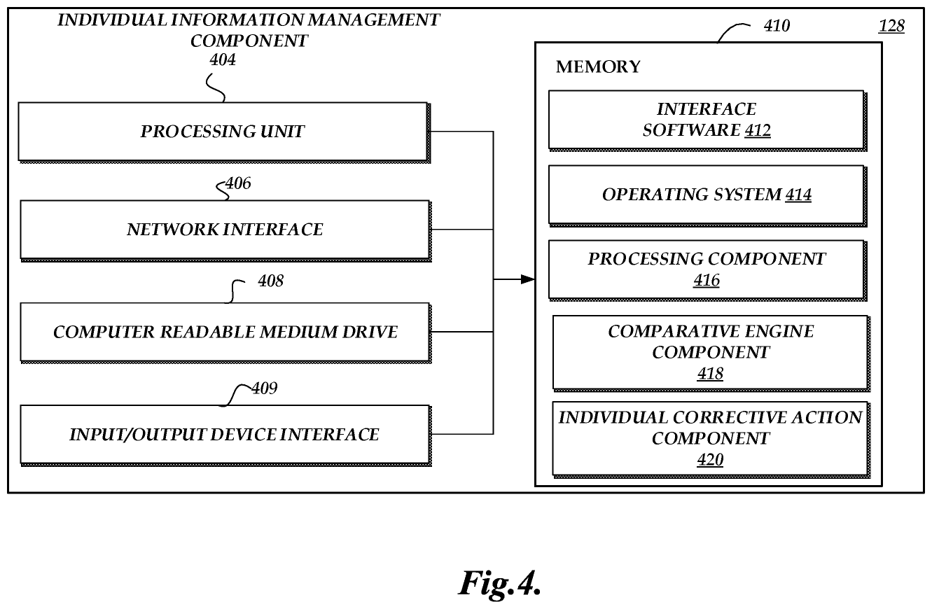

FIG. 4 depicts one embodiment of an architecture of an illustrative server for implementing the individual information management component 128 as described. The general architecture of the individual information management component 128 depicted in FIG. 4 includes an arrangement of computer hardware and software components that may be used to implement aspects of the present disclosure. As illustrated, the individual information management component 128 includes a processing unit 404, a network interface 406, a computer readable medium drive 408, and an input/output device interface 409, all of which may communicate with one another by way of a communication bus. The components of the individual information management component 128 may be physical hardware components or implemented in a virtualized environment.

The network interface 406 may provide connectivity to one or more networks or computing systems, such as the network 106 of FIG. 1. The processing unit 404 may thus receive information and instructions from other computing systems or services via a network. The processing unit 404 may also communicate to and from memory 410 and further provide output information for an optional display via the input/output device interface 409. In some embodiments, the individual information management component 128 may include more (or fewer) components than those shown in FIG. 4.

The memory 410 may include computer program instructions that the processing unit 404 executes in order to implement one or more embodiments. The memory 410 generally includes RAM, ROM, or other persistent or non-transitory memory. The memory 410 may store an operating system 414 that provides computer program instructions for use by the processing unit 404 in the general administration and operation of the individual information management component 128. The memory 410 may further include computer program instructions and other information for implementing aspects of the present disclosure. For example, in one embodiment, the memory 410 includes interface software 412 for receiving and processing assessment information, such as current and historical assessment information, thresholding information and the like. The memory 410 may further include computer program instructions and other information for implementing aspects of the present disclosure. For example, in one embodiment, the memory 410 includes interface software 412 for receiving and processing request for comparative analysis of assessments. Additionally, the memory 410 includes a processing component 416 for managing various services utilized by individual information management component 128. The memory 410 includes a comparative engine component 418 for generating comparative assessments of assessments described herein. The comparative engine component 418 may illustratively correspond to machine learning algorithms to obtain inputs, process the inputs and generate characterizations of the presence of or value of physical indicators. The memory 410 can also include an individual corrective action component 420 for determining and implementing corrective actions for an individual based on the results of the comparative assessments, including customized assessments based on user profile information.

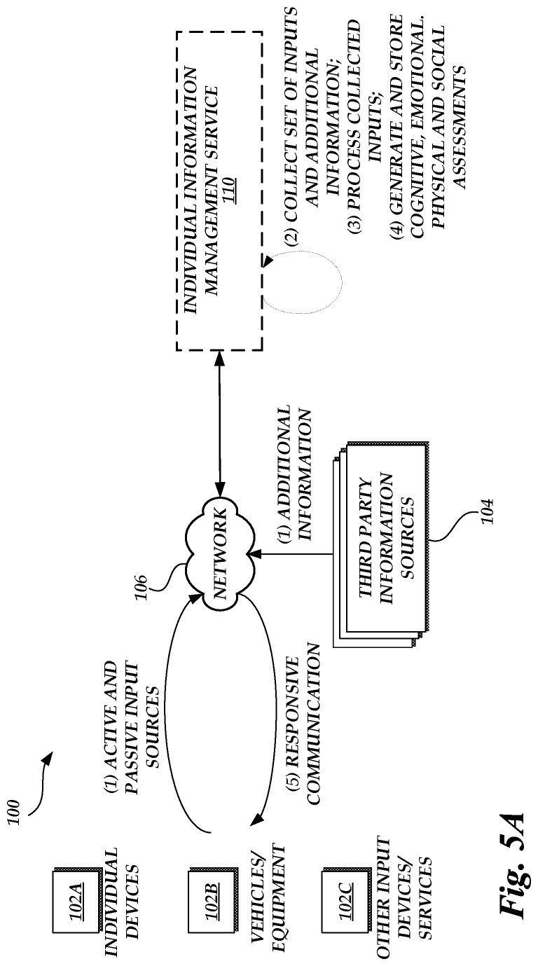

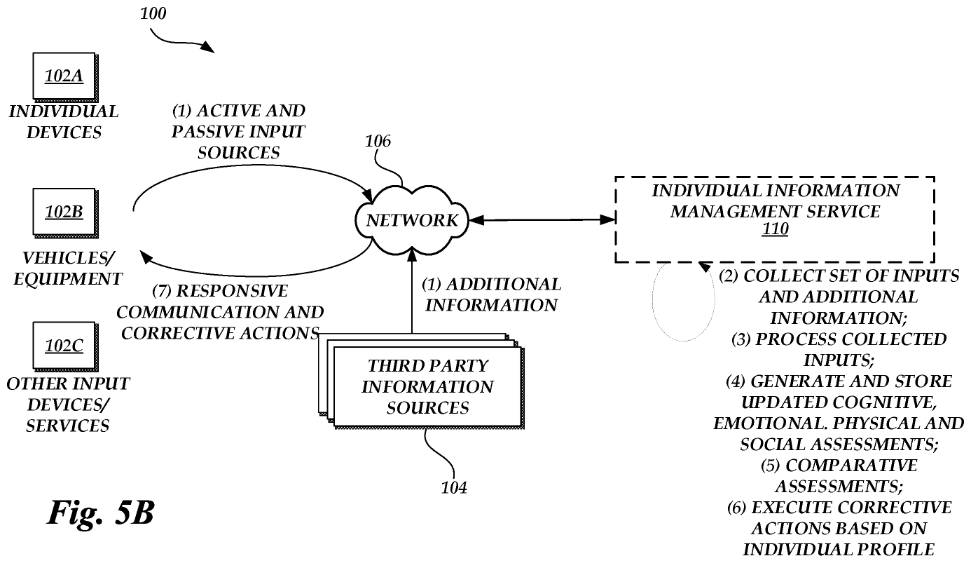

Turning now to FIGS. 5A and 5B, illustrative interactions of the components of the environment 100 will be described. More specifically, FIGS. 5A and 5B illustrate embodiments for the processing of the individual information to generate assessments and identify corrective activities will be described. With reference first to FIG. 5A, at (1), one or more individual devices 102A, 102B, or 102C and one or more third-party information sources 104 collect and provide individual information to the individual information management service 110. Illustratively, the individual information can include passive and active information related to one or more identifiable individuals.

As described previously, the individual information can include various types of information associated with, or otherwise attributable, to one or more identifiable individuals. The information can be further characterized as passive inputs or active inputs based on the type of behavior being exhibited by individuals or the type of information that is collected, or a combination thereof. Illustratively, the passive and active inputs are not necessarily indicative of any of the assessments (e.g., cognitive, emotional, physical or social). However, they can be processed and analyzed in a manner to make assessments.

For example, one set of passive inputs can correspond to information associated with a user device 102A, such as a mobile device that includes or otherwise corresponds to various sensors or input sources. In one aspect, the mobile device can include microphone information that can obtain or measure an individual's audible interaction, such as vocabulary size, language tempo, speech clarity, emotions, voice volume, frequency of conversations, and characteristics of organized speech. For example, individual information can correspond to a processing of conversations heard via a phone, keywords on a calendar like “lunch with X”, terms from social media applications, etc. In another aspect, the mobile device can include speaker/outputs that can measure typical speaker volumes, variations in output volumes, activation of mute, variations in input volumes, and the like. In still further aspects, the mobile device can include accelerometers or other motion sensors that can obtain or measure trembling, dizziness, falls, gait, speed of movement, change in direction, variations in movement and the like. In still further aspects, the mobile device can include interactive controls that can obtain or measure vision or visual cue recognition, spelling or grammar accuracy, concentration, typed vocabulary, errors or corrections and the like. In yet other aspects, the mobile device can obtain or measure location-based services that can obtain or measure location or movement patterns, deviations in from movement, frequency of access of the mobile device and the like. In yet other aspects, the mobile device can obtain or measure image or picture data related to user interaction such as facial expressions, gestures, eye movements, focus and the like.

Active inputs can correspond to information measured from devices as individuals participate in various activities. In one aspect, the active inputs can include information associated with musical instruments or other activities that can measure tempo, accuracy, memory retention, finger manipulation, cue recognition, and the like. For example, the user device 102 can provide GPS or other location-based information. In another aspect, the active inputs can include exercise related measurements, such as distance, power, calories strength measurements, pattern recognition or following, and the like. In still further aspect, active inputs can include health measurements such as heart rate, blood pressure, respiratory rates, distance, sleep measurements, blood oxygen levels, and the like. Still further, the active inputs can include medical history or medical diagnosis information from a third-party information source 104, such as a medical practitioner service. Even further, the active inputs can further include usage data related to the user's manipulation of the user device 102, such as finger manipulation of an instrument. Such inputs can be collected via sensors included in the mobile device (e.g., an instrument) or indirectly by utilizing video or audio sensors to interpret the user interaction. Still further, active inputs can correspond to a series of collected active inputs that may have additional meaning as a set of inputs, in addition to potential meaning as individual inputs or even if the individual inputs may not have independent meaning.

Illustratively, the individual computing user devices 102 and third-party information sources 104 can interact with the individual information management service 110 to provide information in a variety of ways. For example, the individual user devices 102 and third-party information sources 104 can transmit information via an application programming interface (“API”). In other examples, the individual computing devices 102 and third-party information sources 104 can also utilize networking protocols to transmit information, such as scanned information or word processing documents. Still further, the individual information management service 110 can generate various user interfaces, such as a Web page or interactive resource, that can obtain individual information. As previously described, the individual user devices 102 and third-party information sources that generate the individual information may vary based on various criteria, such as availability, age, accessibility and configuration of the individual assessments.

At (2), the individual information management service 110 collects the submitted information. The collection of the individual information may be based on on-demand transmission by the individual computing devices 102 and third-party information sources 104 or based on polling from the individual information management service 110. In other embodiments, if an individual device 102 is processing the data locally, the active and passive input information may not need to be transmitted if collected on the individual device or the individual device may receive transmissions from other individual devices (e.g., short range radio) or third-party information sources 104. At (3), the individual information management service 110 can process the collected information. For example, in a machine-learning environment the individual information management service 110 can process the information to form labels and data in a manner executable by the machine-learned algorithm. In other example, the individual information management service 110 may conduct additional processing to the collected inputs to generate additional scores or metrics, such as applying normalizing, extrapolating, translating or error correcting techniques. Still further, the individual information management service 110 can process information to extract the information utilized in the assessments, such as by parsing social media information, calendaring information, or other communications, or combining individual information for group information.

At (4), the individual information management service 110 generates and stores a plurality of assessments. Such assessments can include cognitive, emotional, physical social, or dietary assessments or various combinations thereof. As described above, individual services of the individual information management service 110 can implement machine-learned algorithms to achieve such assessment. In a supervised training environment, the individual information management service 110 trains the machine learning algorithm to form a machine learned algorithm based on training sets that correspond to processing active and passive inputs and generating outputs associated with assessments. However, by way of non-limiting examples, the machine learning algorithms can incorporate different learning models, including, but not limited to, a supervised learning model, an unsupervised learning model, a reinforcement learning model or a featured learning model. Depending on the type of learning model adopted by the machine learning algorithm, the configuration for processing with the collected individual information can vary (e.g., using a training set for a supervised or semi-supervised learning model). In other embodiments, the machine learning algorithm can implement a reinforcement-based learning model that implements a penalty/reward model determined by the individual information management service 110.

At (5), the machine learned-based algorithm generates a processing result reflective of the selected plurality of assessments, such as selected cognitive, emotional, physical, social and diet assessments. The processing results can include the generation of additional follow up requests or identification of corrective actions. For example, in one embodiment, the individual information management service 110 may utilize individual profile information to select the type of notification, follow up or corrective actions. Still further, the individual information management service 110 may initiate additional communication with a third-party information source 104, such as a medical practitioner to provide the result reflective of the cognitive, emotional, physical, social, and diet assessments or request a set of potential corrective actions reflective of the cognitive, emotional, physical, social, and diet assessments.

Turning now to FIG. 5B, illustratively, the individual information management service 110 can utilize previously generated or collected assessments. At (1), one or more individual devices 102A, 102B, or 102C and one or more third-party information sources 104 provide individual information to the individual information management service 110. Illustratively, the individual information can include passive and active information related to one or more identifiable individuals. The active and passive inputs can correspond to the same inputs previously collected, a subset of the collected inputs or different inputs. Such variations may be based on differences in the availability of the individual computing devices 102 and third-party information sources 104.

As described previously, the individual information can include various types of information associated with, or otherwise attributable, to one or more identifiable individuals. The information can be further characterized as passive inputs or active inputs based on the type of behavior being exhibited by individuals or the type of information that is collected, or a combination thereof. Illustratively, the passive and active inputs are not necessarily indicative of any of the assessments (e.g., cognitive, emotional, physical, social or diet). However, such passive and active inputs can be processed and analyzed in a manner to make desired assessments.

For example, one set of passive inputs can correspond to information associated with a user device 102A, such as a mobile device that various sensors or input sources. In one aspect, the mobile device can include microphone information that can obtain or measure an individual's audible interaction, such as vocabulary size, language tempo, speech clarity, emotions, voice volume, frequency of conversations, and characteristics of organized speech. In another aspect, the mobile device can include speaker/outputs that can measure typical speaker volumes, variations in output volumes, activation of mute, and the like. In still further aspects, the mobile device can include accelerometers or other motion sensors that can obtain or measure trembling, dizziness, falls, gait, speed of movement, change in direction, variations in movement and the like. In still further aspects, the mobile device can include interactive controls that can obtain or measure vision or visual cue recognition, spelling or grammar accuracy, concentration, typed vocabulary, errors or corrections and the like. In yet other aspects, the mobile device can obtain or measure location-based services that can obtain or measure location or movement patterns, deviations in from movement, frequency of access of the mobile device and the like. In yet other aspects, the mobile device can obtain or measure image or picture data related to facial expressions, gestures, eye movements, focus and the like.

Active inputs can correspond to information measured from devices as individuals participate in various activities. In one aspect, the active inputs can include information associated with musical instruments or other activities that can measure tempo, accuracy, memory retention, finger manipulation, cue recognition, and the like. In another aspect, the active inputs can include exercise related measurements, such as distance, power, calories strength measurements, pattern recognition or following, and the like. In still further aspect, active inputs can include health measurements such as heart rate, blood pressure, respiratory rates, distance, sleep measurements, blood oxygen levels, and the like. Still further, the active inputs can include medical history or medical diagnosis information from a third-party information source 104, such as a medical practitioner service. Still further, the active inputs can be verified or correspond to GPS or other location information. Even further, the active inputs can correspond to user manipulation of the mobile device 102 or a measurement of actions measured by the mobile device 102 (e.g., a mobile device monitoring a user manipulation of a separate instrument).

Illustratively, the individual computing devices 102 and third-party information sources 104 can interact with the individual information management service 110 to provide information in a variety of ways. For example, the individual computing devices 102 and third-party information sources 104 can transmit information via an application programming interface (“API”). In other examples, the individual computing devices 102 and third-party information sources 104 can also utilize networking protocols to transmit information, such as scanned information or word processing documents. Still further, the individual information management service 110 can generate various user interfaces, such as a Web page or interactive resource, that can obtain individual information. As previously described, the individual computing devices 102 and third-party information sources that generate the individual information may vary based on various criteria, such as availability, age, accessibility and configuration of the individual assessments.

At (2), the individual information management service 110 collects the submitted information. The collection of the individual information may be based on on-demand transmission by the individual computing devices 102 and third-party information sources 104 or based on polling from the individual information management service 110. Similar to FIG. 5A, in some embodiments, the individual device 102 may process at least some part of the collected individual information. At (3), the individual information management service 110 can process the collected information. For example, in a machine-learning environment the individual information management service 110 can process the information to form labels and data in a manner executable by the machine-learned algorithm. In other example, the individual information management service 110 may conduct additional processing to the collected inputs to generate additional scores or metrics, such as applying normalizing, extrapolating, translating or error correcting techniques.

At (4), the individual information management service 110 generates and stores updated selected assessments, including cognitive, emotional, physical, social, and diet assessments, or a combination thereof. As described above, individual services of the individual information management service 110 can implement machine-learned algorithms to achieve such assessment. In a supervised training environment, the individual information management service 110 trains the machine learning algorithm to form a machine learned algorithm based on training sets that correspond to processing active and passive inputs and generating outputs associated with assessments. However, by way of non-limiting examples, the machine learning algorithms can incorporate different learning models, including, but not limited to, a supervised learning model, an unsupervised learning model, a reinforcement learning model or a featured learning model. Depending on the type of learning model adopted by the machine learning algorithm, the configuration for processing with the collected individual information can vary (e.g., using a training set for a supervised or semi-supervised learning model). In other embodiments, the machine learning algorithm can implement a reinforcement-based learning model that implements a penalty/reward model determined by the individual information management service 110.

At (5), the individual information management service 110 can then make comparative assessments of the currently calculated assessments and historical assessment information. Illustratively, the individual information management service 110 can compare the current and historical determine assessments across each type of assessment (cognitive, emotional, physical, social or diet) and identify similarities and differences. The individual information management service 110 can then obtain thresholds that establishes whether such similarities or differences can be determined to be significant. The thresholds can illustratively be based on the type of assessment type, age of the individual, manual selection, administrator or medical personnel configuration, and the like. For example, the individual information management service 110 can be configured with a threshold that indicates any indication of memory loss may be considered significant compared with a larger threshold for indications of variations in mood swings assessments. In another example, the individual information management service 110 can be configured to adjust thresholds based on regional or cultural criteria, such as analysis of speech patterns, vocabulary usage or analysis, and the like.

At (6), the individual information management service 110 executes one or more mitigation or corrective actions reflective of the cognitive, emotional, physical and social assessments and comparative analysis. The processing results can include the generation of additional follow up requests or identification of corrective actions. Illustratively, the corrective actions can be configured by the individual information management service 110 based on the comparative analysis, such as the level or severity of corrective actions may be dependent on the threshold determination or type of assessment. For example, the individual information management service 110 can generate notifications to individuals, such as health care providers, cause modification of the computing devices 102, provide the individuals with alerts and the like, such as illustrated at (7). Still further, in some embodiments, the identification of the corrective actions can be based, at least in part, on customized medical diagnosis or customized corrective actions plans for an individual or set of individuals. In this regard, the individual information management service 110 may interact with third-party information sources 104 to obtain more information or otherwise access personal medical history information in a manner to obtain corrective action information. Still further in other embodiments, the mitigation techniques can include an assessment that captures how the individual interacts with the computing devices 102 to facilitate the instrumentation of such interaction. For example, the individual management service 110 may identify specific patterns of interaction (e.g., a mobile device or an instrument) and define the pattern as part of the various assessments.

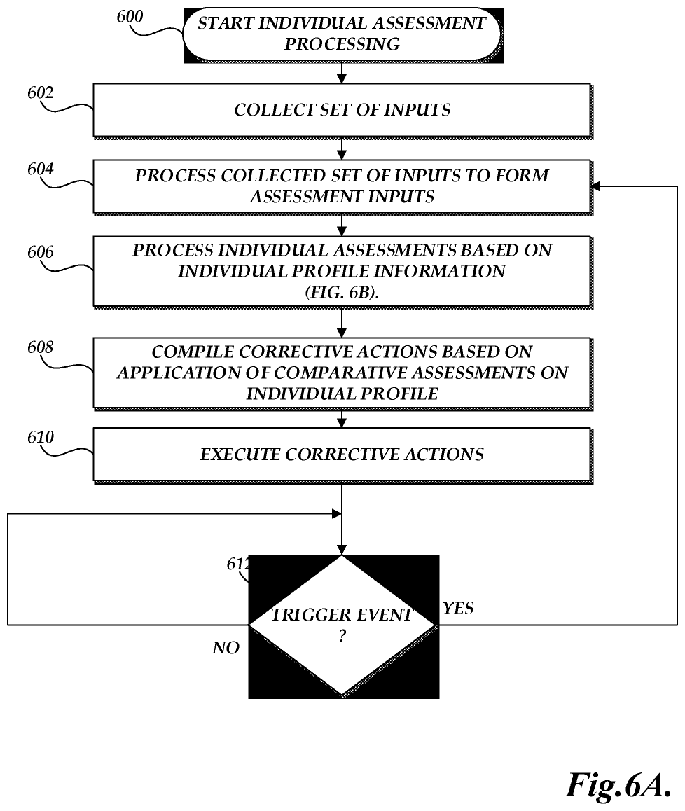

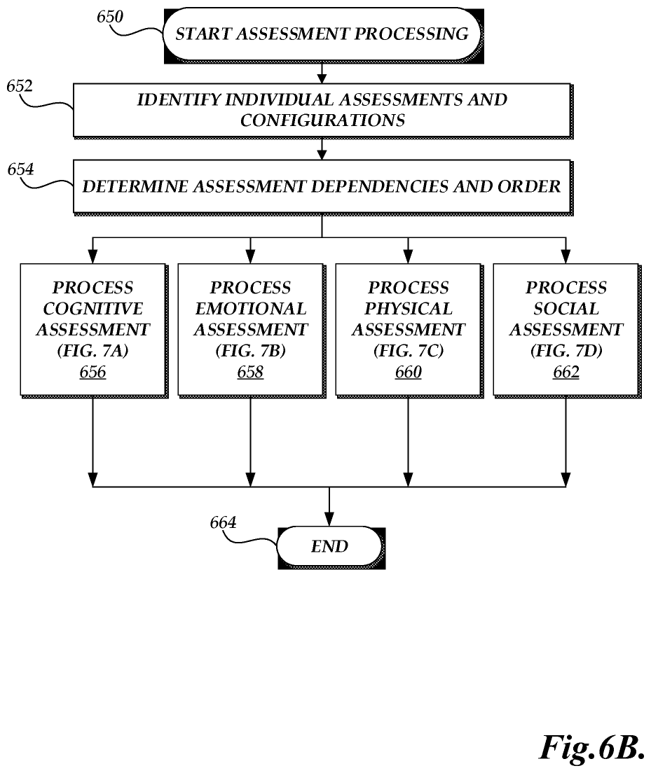

Turning now to FIGS. 6A and 6B, a routine 600 for facilitating individual assessments will be described. Routine 600 is illustratively implemented by the individual information management service 110. At block 602, the individual information management service 110 collects information from one or more individual devices 102A, 102B, or 102C and one or more third-party information sources 104 provide individual information to the individual information management service 110. Illustratively, the individual information can include passive and active information related to one or more identifiable individuals. The active and passive inputs can correspond to the same inputs previously collected, a subset of the collected inputs or different inputs. Such variations may be based on different criteria, such as differences in the availability of the individual computing devices 102 and third-party information sources 104, accessibility of the data, type of assessments being conducts, configuration or preferences of the individual, and the like.

As described previously, the individual information can include various types of information associated with, or otherwise attributable, to one or more identifiable individuals. The information can be further characterized as passive inputs or active inputs based on the type of behavior being exhibited by individuals or the type of information that is collected, or a combination thereof. Illustratively, the passive and active inputs are not necessarily indicative of any of the assessments (e.g., cognitive, emotional, physical, social, diet). But, they can be processed and analyzed in a manner to make assessments.

For example, one set of passive inputs can correspond to information associated with a user device 102A, such as a mobile device that various sensors or input sources. In one aspect, the mobile device can include microphone information that can obtain or measure an individual's audible interaction, such as vocabulary size, language tempo, speech clarity, emotions, voice volume, frequency of conversations, and characteristics of organized speech. In another aspect, the mobile device can include speaker/outputs that can measure typical speaker volumes, variations in output volumes, activation of mute, and the like. In still further aspects, the mobile device can include accelerometers or other motion sensors that can obtain or measure trembling, dizziness, falls, gait, speed of movement, change in direction, variations in movement and the like. In still further aspects, the mobile device can include interactive controls that can obtain or measure vision or visual cue recognition, spelling or grammar accuracy, concentration, typed vocabulary, errors or corrections and the like. In yet other aspects, the mobile device can obtain or measure location-based services that can obtain or measure location or movement patterns, deviations in from movement, frequency of access of the mobile device and the like. In yet other aspects, the mobile device can obtain or measure image or picture data related to facial expressions, gestures, eye movements, focus and the like.

Active inputs can correspond to information measured from devices as individuals participate in various activities. In one aspect, the active inputs can include information associated with musical instruments or other activities that can measure tempo, accuracy, memory retention, finger manipulation, cue recognition, and the like. In another aspect, the active inputs can include exercise related measurements, such as distance, power, calories strength measurements, pattern recognition or following, and the like. In still further aspect, active inputs can include health measurements such as heart rate, blood pressure, respiratory rates, distance, sleep measurements, blood oxygen levels, and the like.

Illustratively, the individual computing devices 102 and third-party information sources 104 can interact with the individual information management service 110 to provide information in a variety of ways. For example, the individual computing devices 102 and third-party information sources 104 can transmit information via an application programming interface (“API”). In other examples, the individual computing devices 102 and third-party information sources 104 can also utilize networking protocols to transmit information, such as scanned information or word processing documents. Still further, the individual information management service 110 can generate various user interfaces, such as a Web page or interactive resource, that can obtain individual information.

At block 604, the individual information management service 110 can process the collected information. For example, in a machine-learning environment the individual information management service 110 can process the information to form labels and data in a manner executable by the machine-learned algorithm. In other example, the individual information management service 110 may conduct additional processing to the collected inputs to generate additional scores or metrics, such as applying normalizing, extrapolating, translating or error correcting techniques.

At block 606, the individual information management service 110 generates and stores updated cognitive, emotional, physical and social assessments. As described above, individual services of the individual information management service 110 can implement machine-learned algorithms to achieve such assessment. In a supervised training environment, the individual information management service 110 trains the machine learning algorithm to form a machine learned algorithm based on training sets that correspond to processing active and passive inputs and generating outputs associated with assessments. However, by way of non-limiting examples, the machine learning algorithms can incorporate different learning models, including, but not limited to, a supervised learning model, an unsupervised learning model, a reinforcement learning model or a featured learning model. Depending on the type of learning model adopted by the machine learning algorithm, the configuration for processing with the collected individual information can vary (e.g., using a training set for a supervised or semi-supervised learning model). In other embodiments, the machine learning algorithm can implement a reinforcement-based learning model that implements a penalty/reward model determined by the individual information management service 110.

As previously described, the individual information management service 110 can then make comparative assessments of the currently calculated assessments and historical assessment information. Illustratively, the individual information management service 110 can compare the current and historical determine assessments across each type of assessment (cognitive, emotional, physical, social, or diet) and identify similarities and differences. The individual information management service 110 can then obtain thresholds that establishes whether such similarities or differences can be determined to be significant. The thresholds can illustratively be based on the type of assessment type, age of the individual, manual selection and the like. For example, the individual information management service 110 can be configured with a threshold that indicates any indication of memory loss may be considered significant compared with a larger threshold for indications of variations in mood swings assessments. Accordingly, at block 608, the individual information management service 110 identifies or compiles the set of corrective actions and executes the corrective actions at block 610.

Illustratively, the individual information management service 110 executes one or more corrective actions reflective of the cognitive, emotional, physical and social assessments and comparative analysis. The processing results can include the generation of additional follow up requests or identification of corrective actions. Illustratively, the corrective actions can be configured by the individual information management service 110 based on the comparative analysis, such as the level or severity of corrective actions may be dependent on the threshold determination or type of assessment. For example, the individual information management service 110 can generate notifications to individuals, such as health care providers, cause modification of the computing devices 102, provide the individuals with alerts and the like. Still further, in some embodiments, the identification of the corrective actions can be based, at least in part, on customized medical diagnosis or customized corrective actions plans for an individual or set of individuals. In this regard, the individual information management service 110 may interact with third-party information sources 104 to obtain more information or otherwise access personal medical history information in a manner to obtain corrective action information. In some embodiments, the individual information management service 110 can apply financial-based selection criteria that prioritizes or selects corrective actions based on an attributed cost to the corrective action. In this embodiment, the financial-based selection criteria may be unique to an individual or set of individuals, such as personal preferences, insurance information, and the like.

At decision block 612, the individual information management service 110 then determines whether there is a trigger event to make additional assessments. Illustratively, the trigger events can be based on time frequency (e.g., conduct assessments and analysis every month), on availability of input data, manual input (such as health care workers or individuals), and the like. If no trigger event is detected, the routine 600 remains at block 618. Once a trigger event is detected, the routine 600 returns to block 604 to process collected inputs.

With reference to FIG. 6B, a sub-routine 650 related to the processing one or more assessments based on collected and processing individual information will be described. Sub-routine 650 may be illustratively implemented by the individual information management service 110, such via the assessment controller 111. Sub-routine 650 may be illustratively implement in a manner such as for the processing of individual assessments in block 606 (FIG. 6A).

At block 652, the individual information management service 110 identifies one or more individual assessments and configurations for each identified individual assessment. As described above, the individual information management service 110 generates and stores updated cognitive, emotional, physical, social and diet assessments through the utilization of one or more assessment services/components. Each individual assessment 110 may be configured differently based on individual specific configurations, group configurations, service provider preferences, and the like. At block 654, the individual information management service 110 determines dependencies and order of execution for each of the identified assessments. Illustratively, the individual information management service 110 may be configured or otherwise determine dependencies in the generation of assessments. For example, the determination of an emotional assessment may require or consider one or more outputs associated with cognitive assessments. Accordingly, the individual information management service 110 would typically cause the generation and completion of the cognitive assessment prior to initiating the emotional assessment. In other embodiments, the individual information management service 110 may determine to process two or more assessments in parallel to facilitate completion, especially for assessments that are not dependent or in which the dependency can be ignored or mitigated.

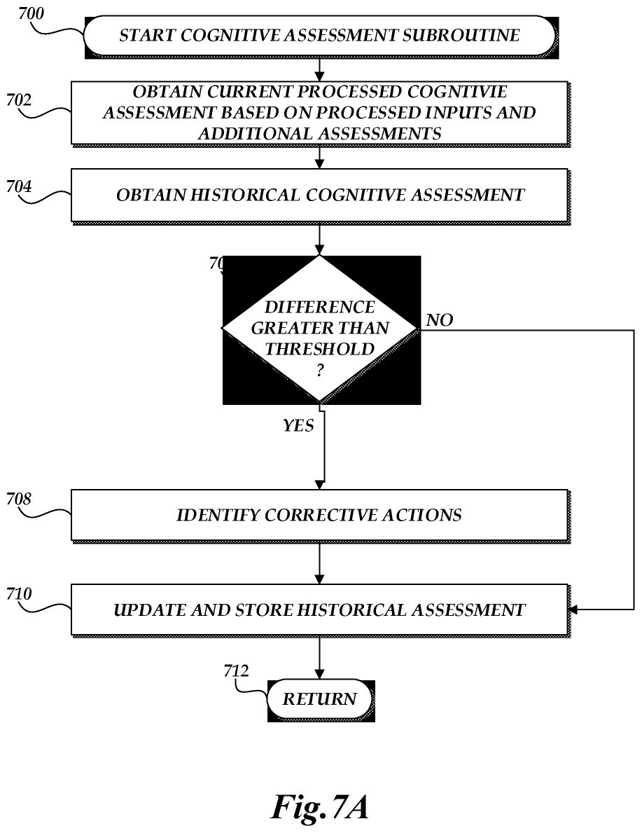

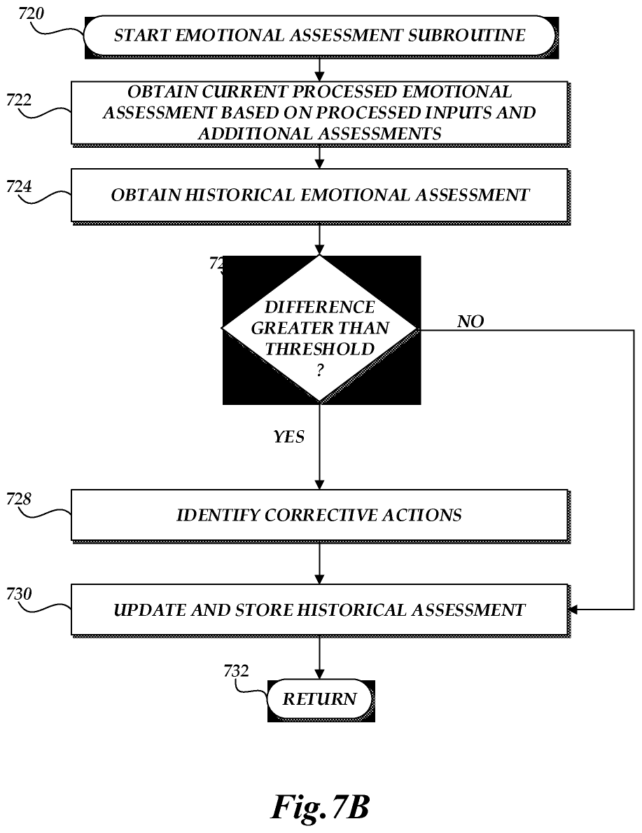

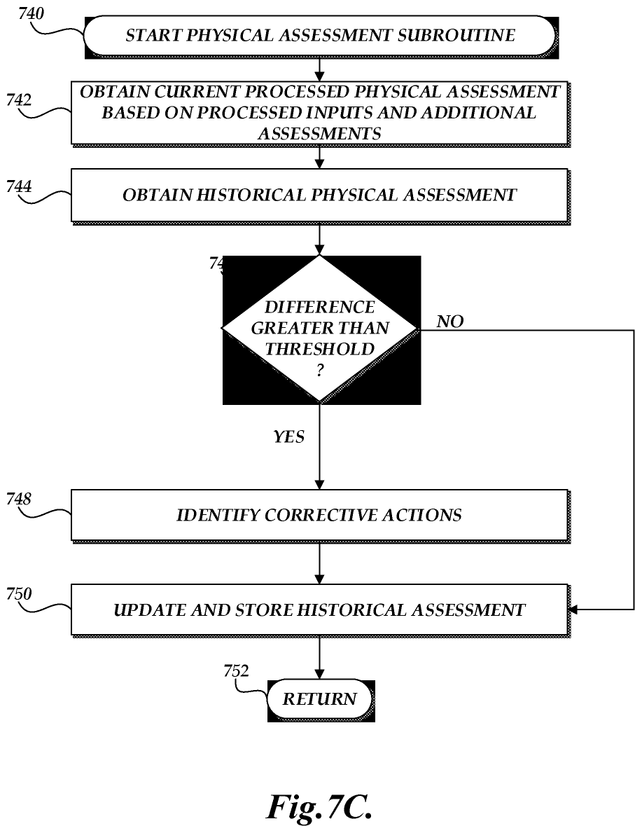

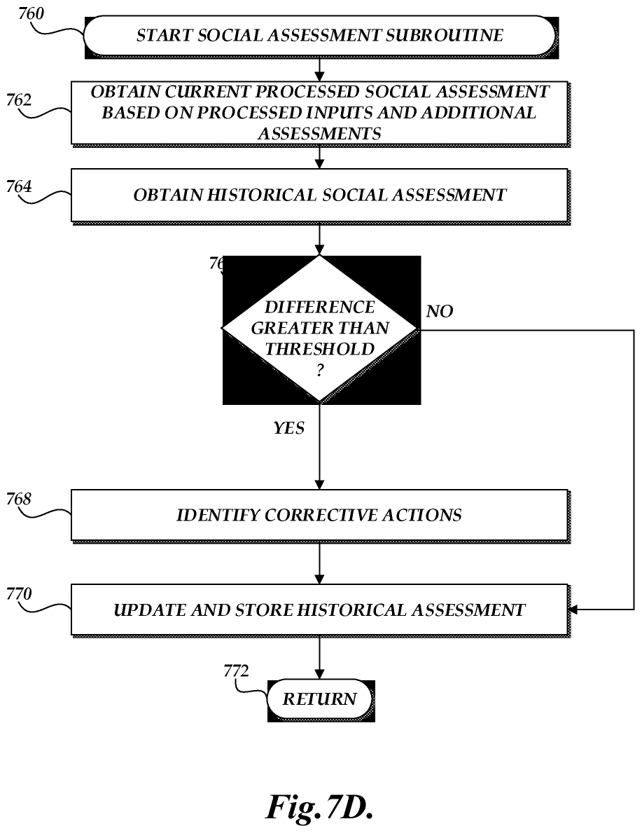

FIG. 6B illustrates the processing of a set of assessments in parallel. At block 656, the individual information management service 110 processes cognitive assessments. At block 658, the individual information management service 110 processes emotional assessments. At block 660, the individual information management service 110 processes physical assessments. At block 662, the individual information management service 110 processes social assessments. Finally, at block 663, the individual information management service processes diet assessments. Individual sub-routines for blocks 656-664 will be described with regard to FIGS. 7A-7E. In other embodiments, two or more assessments may be completed in a serial manner such that the completion of a preceding assessment be required prior to completion of the next assessment. At block 665, the sub-routine 650 returns.

Turning now to FIGS. 7A-7E, sub-routines for conducting assessments and comparative analysis for cognitive, emotional, physical and social assessments will be described. With reference first to FIG. 7A, a cognitive assessment subroutine 700 will be described. At block 702, the individual information management service 110 obtains a current cognitive assessment. As described above, the generation of cognitive assessment may be based on applying machine learned or machine learning algorithms on a set of input data to determine one or more characteristics of cognitive assessments. Examples of such cognitive assessments can include, but is not limited to a determination or characterization of confusion, attention, decision-making, visual special problems and memory loss. At block 704, the individual information management service 110 obtains historical information relate dot previously calculated or determined cognitive assessments.

At decision block 706, a test is conducted to determine whether any identified differences or variations exceeds a threshold. As described above, the individual information management service 110 can further implement a comparative engine or process to compare a current plurality of assessment to historical assessments. In this regard, the individual information management service can compare potential differences or deviations against thresholds. Individual thresholds may be set based on age-based criteria, such that the individual information management service can apply different threshold based on age or other information. Additionally, the individual information management service can apply different types of thresholds based on the type of assessment (e.g., cognitive, emotional, physical or social). Still further, the individual information management service can further include some dynamic thresholds that can be adjusted based on manual inputs (e.g., health care provider), or based on historical measurable differences for individuals.

If the determined differences exceed a threshold, at block 708, the individual information management service 110 then identifies corrective actions related to the determined exceed threshold as described above. Alternatively, or after the identified corrective actions at block 708, the individual information management service 110 updates and stores historical cognitive assessment information and sub-routine 700 returns at block 712.

With reference first to FIG. 7A, a cognitive assessment subroutine 700 will be described. At block 702, the individual information management service 110 obtains a current cognitive assessment. As described above, the generation of cognitive assessment may be based on applying machine learned or machine learning algorithms on a set of input data to determine one or more characteristics of cognitive assessments. Examples of such cognitive assessments can include, but is not limited to a determination or characterization of confusion, attention, decision-making, visual special problems and memory loss. At block 704, the individual information management service 110 obtains historical information related to previously calculated or determined cognitive assessments.

At decision block 706, a test is conducted to determine whether any identified differences or variations exceeds a threshold. As described above, the individual information management service 110 can further implement a comparative engine or process to compare a current plurality of assessment to historical assessments. In this regard, the individual information management service can compare potential differences or deviations against thresholds. Individual thresholds may be set based on age-based criteria, such that the individual information management service can apply different threshold based on age or other information. Additionally, the individual information management service can apply different types of thresholds based on the type of assessment (e.g., cognitive, emotional, physical or social). Still further, the individual information management service can further include some dynamic thresholds that can be adjusted based on manual inputs (e.g., health care provider), or based on historical measurable differences for individuals.

If the determined differences exceed a threshold, at block 708, the individual information management service 110 then identifies corrective actions related to the determined exceed threshold as described above. Alternatively, or after the identified corrective actions at block 708, the individual information management service 110 updates and stores historical cognitive assessment information and sub-routine 700 returns at block 712.

With reference next to FIG. 7B, an emotional assessment subroutine 720 will be described. At block 722, the individual information management service 110 obtains a current emotional assessment. As described above, the generation of emotional assessment may be based on applying machine learned or machine learning algorithms on a set of input data to determine one or more characteristics of emotional assessments. Examples of such emotional assessments includes, but is not limited to, a determination or characterization of depression, apathy, personality changes, mood swings or variations and agitation. At block 724, the individual information management service 110 obtains historical information related to previously calculated or determined emotional assessments.

At decision block 726, a test is conducted to determine whether any identified differences or variations exceeds a threshold. As described above, the individual information management service 110 can further implement a comparative engine or process to compare a current plurality of assessment to historical assessments. In this regard, the individual information management service can compare potential differences or deviations against thresholds. Individual thresholds may be set based on age-based criteria, such that the individual information management service can apply different threshold based on age or other information. Additionally, the individual information management service can apply different types of thresholds based on the type of assessment (e.g., cognitive, emotional, physical, social or diet). Still further, the individual information management service can further include some dynamic thresholds that can be adjusted based on manual inputs (e.g., health care provider), or based on historical measurable differences for individuals.

If the determined differences exceed a threshold, at block 728, the individual information management service 110 then identifies corrective actions related to the determined exceed threshold as described above. Alternatively, or after the identified corrective actions at block 728, the individual information management service 110 updates and stores historical emotional assessment information and sub-routine 720 returns at block 732.

With reference next to FIG. 7C, a physical assessment subroutine 740 will be described. At block 742, the individual information management service 110 obtains a current physical assessment. As described above, the generation of physical assessment may be based on applying machine learned or machine learning algorithms on a set of input data to determine one or more characteristics of physical assessments. Examples of such physical assessments can include, but are not limited to, a determination or characterization of visual hallucinations, auditory hallucinations, olfactory hallucinations, tactile hallucinations, eye movement, balance, falls, facial expression, reduced strength, reflexes, muscle stiffness, muscle contracture, jerking, tremors, gait, REM sleep, drowsiness, staring, disorganized speech, incoordination, posture, mispronunciation, non-verbal gesturing and hearing/visual skills. At block 744, the individual information management service 110 obtains historical information related to previously calculated or determined physical assessments.

At decision block 746, a test is conducted to determine whether any identified differences or variations exceeds a threshold. As described above, the individual information management service 110 can further implement a comparative engine or process to compare a current plurality of assessment to historical assessments. In this regard, the individual information management service can compare potential differences or deviations against thresholds. Individual thresholds may be set based on age-based criteria, such that the individual information management service can apply different threshold based on age or other information. Additionally, the individual information management service can apply different types of thresholds based on the type of assessment (e.g., cognitive, emotional, physical, social or diet). Still further, the individual information management service can further include some dynamic thresholds that can be adjusted based on manual inputs (e.g., health care provider), or based on historical measurable differences for individuals.

If the determined differences exceed a threshold, at block 748, the individual information management service 110 then identifies corrective actions related to the determined exceed threshold as described above. Alternatively, or after the identified corrective actions at block 748, the individual information management service 110 updates and stores historical emotional assessment information and sub-routine 740 returns at block 752.

With reference next to FIG. 7D, a social assessment subroutine 760 will be described. At block 762, the individual information management service 110 obtains a current social assessment. As described above, the generation of social assessment may be based on applying machine learned or machine learning algorithms on a set of input data to determine one or more characteristics of social assessments. Examples of such social assessments can include, but are not limited to, a determination of depression, reduced social interaction, withdrawn and sedentariness, confusion, attention, decision-making, visual special problems and memory loss.

At block 764, the individual information management service 110 obtains historical information related to previously calculated or determined social assessments.

At decision block 766, a test is conducted to determine whether any identified differences or variations exceeds a threshold. As described above, the individual information management service 110 can further implement a comparative engine or process to compare a current plurality of assessment to historical assessments. In this regard, the individual information management service can compare potential differences or deviations against thresholds. Individual thresholds may be set based on age-based criteria, such that the individual information management service can apply different threshold based on age or other information. Additionally, the individual information management service can apply different types of thresholds based on the type of assessment (e.g., cognitive, emotional, physical, social or diet). Still further, the individual information management service can further include some dynamic thresholds that can be adjusted based on manual inputs (e.g., health care provider), or based on historical measurable differences for individuals.

If the determined differences exceed a threshold, at block 768, the individual information management service 110 then identifies corrective actions related to the determined exceed threshold as described above. Alternatively, or after the identified corrective actions at block 768, the individual information management service 110 updates and stores historical social assessment information and sub-routine 760 returns at block 772.

At least some elements of a device of the present application can be controlled—and at least some steps of a method of the invention can be effectuated, in operation—with a programmable processor governed by instructions stored in a memory. The memory may be random access memory (RAM), read-only memory (ROM), flash memory or any other memory, or combination thereof, suitable for storing control software or other instructions and data. Those skilled in the art should also readily appreciate that instructions or programs defining the functions of the present invention may be delivered to a processor in many forms, including, but not limited to, information permanently stored on non-writable storage media (e.g. read-only memory devices within a computer, such as ROM, or devices readable by a computer I/O attachment, such as CD-ROM or DVD disks), information alterably stored on writable storage media (e.g. floppy disks, removable flash memory and hard drives) or information conveyed to a computer through communication media, including wired or wireless computer networks. In addition, while the invention may be embodied in software, the functions necessary to implement the invention may optionally or alternatively be embodied in part or in whole using firmware and/or hardware components, such as combinatorial logic, Application Specific Integrated Circuits (ASICs), Field-Programmable Gate Arrays (FPGAs) or other hardware or some combination of hardware, software and/or firmware components.

As used herein, a phrase referring to “at least one of” a list of items refers to any combination of those items, including single members. As an example, “at least one of: a, b, or c” is intended to cover: a, b, c, a-b, a-c, b-c, and a-b-c.

Various modifications to the implementations described in this disclosure may be readily apparent to those skilled in the art, and the generic principles defined herein may be applied to other implementations without departing from the spirit or scope of this disclosure. Thus, the claims are not intended to be limited to the implementations shown herein, but are to be accorded the widest scope consistent with this disclosure, the principles and the novel features disclosed herein.

Certain features that are described in this specification in the context of separate implementations also can be implemented in combination in a single implementation. Conversely, various features that are described in the context of a single implementation also can be implemented in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Depending on the embodiment, certain acts, events, or functions of any of the processes or algorithms described herein can be performed in a different sequence, can be added, merged, or left out altogether (e.g., not all described operations or events are necessary for the practice of the algorithm). Moreover, in certain embodiments, operations or events can be performed concurrently, e.g., through multi-threaded processing, interrupt processing, or multiple processors or processor cores or on other parallel architectures, rather than sequentially.

The various illustrative logical blocks, modules, routines, and algorithm steps described in connection with the embodiments disclosed herein can be implemented as electronic hardware, or as a combination of electronic hardware and executable software. To clearly illustrate this interchangeability, various illustrative components, blocks, modules, and steps have been described above generally in terms of their functionality. Whether such functionality is implemented as hardware, or as software that runs on hardware, depends upon the particular application and design constraints imposed on the overall system. The described functionality can be implemented in varying ways for each particular application, but such implementation decisions should not be interpreted as causing a departure from the scope of the disclosure.

Moreover, the various illustrative logical blocks and modules described in connection with the embodiments disclosed herein can be implemented or performed by a machine, such as a machine learning service server, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A machine learning service server can be or include a microprocessor, but in the alternative, the machine learning service server can be or include a controller, microcontroller, or state machine, combinations of the same, or the like configured to generate and publish machine learning services backed by a machine learning model. A machine learning service server can include electrical circuitry configured to process computer-executable instructions. Although described herein primarily with respect to digital technology, a machine learning service server may also include primarily analog components. For example, some or all of the modeling, simulation, or service algorithms described herein may be implemented in analog circuitry or mixed analog and digital circuitry. A computing environment can include any type of computer system, including, but not limited to, a computer system based on a microprocessor, a mainframe computer, a digital signal processor, a portable computing device, a device controller, or a computational engine within an appliance, to name a few.

The elements of a method, process, routine, or algorithm described in connection with the embodiments disclosed herein can be embodied directly in hardware, in a software module executed by a machine learning service server, or in a combination of the two. A software module can reside in RAM memory, flash memory, ROM memory, EPROM memory, EEPROM memory, registers, hard disk, a removable disk, a CD-ROM, or any other form of a non-transitory computer-readable storage medium. An illustrative storage medium can be coupled to the machine learning service server such that the machine learning service server can read information from, and write information to, the storage medium. In the alternative, the storage medium can be integral to the machine learning service server. The machine learning service server and the storage medium can reside in an ASIC. The ASIC can reside in a user terminal. In the alternative, the machine learning service server and the storage medium can reside as discrete components in a user terminal (e.g., access device or network service client device).

Conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without other input or prompting, whether these features, elements and/or steps are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list.

Disjunctive language such as the phrase “at least one of X, Y, or Z,” unless specifically stated otherwise, is otherwise understood with the context as used in general to present that an item, term, etc., may be either X, Y, or Z, or any combination thereof (e.g., X, Y, and/or Z). Thus, such disjunctive language is not generally intended to, and should not, imply that certain embodiments require at least one of X, at least one of Y, or at least one of Z to each be present.

Unless otherwise explicitly stated, articles such as “a” or “an” should generally be interpreted to include one or more described items. Accordingly, phrases such as “a device configured to” are intended to include one or more recited devices. Such one or more recited devices can also be collectively configured to carry out the stated recitations. For example, “a processor configured to carry out recitations A, B and C” can include a first processor configured to carry out recitation A working in conjunction with a second processor configured to carry out recitations B and C.

As used herein, the terms “determine” or “determining” encompass a wide variety of actions. For example, “determining” may include calculating, computing, processing, deriving, looking up (e.g., looking up in a table, a database or another data structure), ascertaining and the like. Also, “determining” may include receiving (e.g., receiving information), accessing (e.g., accessing data in a memory) and the like. Also, “determining” may include resolving, selecting, choosing, establishing, and the like.

As used herein, the term “selectively” or “selective” may encompass a wide variety of actions. For example, a “selective” process may include determining one option from multiple options. A “selective” process may include one or more of: dynamically determined inputs, preconfigured inputs, or user-initiated inputs for making the determination. In some embodiments, an n-input switch may be included to provide selective functionality where n is the number of inputs used to make the selection.

As used herein, the terms “provide” or “providing” encompass a wide variety of actions. For example, “providing” may include storing a value in a location for subsequent retrieval, transmitting a value directly to the recipient, transmitting or storing a reference to a value, and the like. “Providing” may also include encoding, decoding, encrypting, decrypting, validating, verifying, and the like.

As used herein, the term “message” encompasses a wide variety of formats for communicating (e.g., transmitting or receiving) information. A message may include a machine readable aggregation of information such as an XML document, fixed field message, comma separated message, or the like. A message may, in some embodiments, include a signal utilized to transmit one or more representations of the information. While recited in the singular, it will be understood that a message may be composed, transmitted, stored, received, etc. in multiple parts.

As used herein, the term “correspond” encompasses a range of relative relationships between two or more elements. Correspond may refer to equality (e.g., match). Correspond may refer to partial-equality (e.g., partial match, fuzzy match, soundex). Correspond may refer to a value which falls within a range of values.

As used herein “receive” or “receiving” may include specific algorithms for obtaining information. For example, receiving may include transmitting a request message for the information. The request message may be transmitted via a network as described above. The request message may be transmitted according to one or more well-defined, machine readable standards which are known in the art. The request message may be stateful in which case the requesting device and the device to which the request was transmitted maintain a state between requests. The request message may be a stateless request in which case the state information for the request is contained within the messages exchanged between the requesting device and the device serving the request. One example of such state information includes a unique token that can be generated by either the requesting or serving device and included in messages exchanged. For example, the response message may include the state information to indicate what request message caused the serving device to transmit the response message.

As used herein “generate” or “generating” may include specific algorithms for creating information based on or using other input information. Generating may include retrieving the input information such as from memory or as provided input parameters to the hardware performing the generating. Once obtained, the generating may include combining the input information. The combination may be performed through specific circuitry configured to provide an output indicating the result of the generating. The combination may be dynamically performed such as through dynamic selection of execution paths based on, for example, the input information, device operational characteristics (e.g., hardware resources available, power level, power source, memory levels, network connectivity, bandwidth, and the like). Generating may also include storing the generated information in a memory location. The memory location may be identified as part of the request message that initiates the generating. In some embodiments, the generating may return location information identifying where the generated information can be accessed. The location information may include a memory location, network locate, file system location, or the like.

As used herein a “user interface” (also referred to as an interactive user interface, a graphical user interface or a UI) may refer to a network-based interface including data fields and/or other controls for receiving input signals or providing electronic information and/or for providing information to the user in response to any received input signals. A UI may be implemented in whole or in part using technologies such as hyper-text mark-up language (HTML), FLASH™, JAVA™, .NET™, web services, and rich site summary (RSS). In some embodiments, a UI may be included in a stand-alone client (for example, thick client, fat client) configured to communicate (e.g., send or receive data) in accordance with one or more of the aspects described.

While the above detailed description has shown, described, and pointed out novel features as applied to various embodiments, it can be understood that various omissions, substitutions, and changes in the form and details of the devices or algorithms illustrated can be made without departing from the spirit of the disclosure. As can be recognized, certain embodiments described herein can be embodied within a form that does not provide all of the features and benefits set forth herein, as some features can be used or practiced separately from others. The scope of certain embodiments disclosed herein is indicated by the appended claims rather than by the foregoing description. All changes which come within the meaning and range of equivalency of the claims are to be embraced within their scope.

What is claimed is:

1. A system comprising:
a plurality of assessment data stores, wherein individual assessment data stores are configured to maintain information related to machine learned algorithms for conducting an assessment;
a plurality of user profile data stores configured to maintain at least historical information related to previously conducted user assessments; and
a processor in communication with the data store, wherein the processor is configured with specific computer-executable instructions to perform operations including:
obtaining a set of passive and active inputs corresponding to user interactions with one or more devices;
processing the set of passive and active inputs according to machine learned algorithm configured to generate a cognitive assessment;
processing at least one of the set of passive and active inputs and an additional assessment according to machine learned algorithm configured to generate an emotional assessment;
processing at least one of the set of passive and active inputs and an additional assessment according to machine learned algorithm configured to generate a physical assessment;
processing at least one of the set of passive and active inputs and an additional assessment according to machine learned algorithm configured to generate a social assessment;
storing the generated cognitive, emotional, physical and social assessments; and
generating at least one processing result corresponding to at least one of the generated cognitive, emotional, physical and social assessment.

2. The system of claim 1, wherein the operations further include processing the cognitive, emotional, physical and social assessments in parallel.

3. The system of claim 1, wherein the processing result includes at least one notification indicative of the generated cognitive, emotional, physical and social assessments.

4. The system of claim 1, wherein the operations further include generating at least one additional interaction based on the generated cognitive, emotional, physical and social assessments.

5. The system of claim 1, wherein the machine learned algorithms for conducting an assessment correspond to age-based ranges, the operations further include selecting individual machine learned algorithms based on age information associated with the set of passive and active inputs.

6. A computer-implemented method comprising:
obtaining a set of passive and active inputs corresponding to user interactions with one or more devices;
processing at least one of the set of passive and active inputs and additional assessment according to a plurality of machine learned algorithm configured to generate a set of individual assessments; and
generating at least one processing result corresponding to the set of individual assessments.

7. The computer-implemented method of claim 6, wherein the set of passive and active inputs corresponds to a plurality of devices, wherein individual devices are configured to provide at least one of an active or passive input.

8. The computer-implemented method of claim 6 further comprising processing data from the one or more devices to generate passive input data.

9. The computer-implemented method of claim 6 further comprising processing the plurality of assessments in parallel.

10. The computer-implemented method of claim 6, wherein at least one assessment is dependent on an assessment, the method further comprising processing the plurality of assessment in an ordered manner based on dependencies.

11. The computer-implemented method of claim 6, wherein generating the processing result includes at least one notification indicative of at least one of generated cognitive, emotional, physical social, or diet assessments.

12. The computer-implemented method of claim 6, wherein generating the processing result includes generating at least one additional interaction based on the set assessment.

13. The computer-implemented method of claim 6, wherein the machine learned algorithms for conducting an assessment correspond to age-based ranges, and wherein processing at least one of the set of passive and active inputs and additional assessment according to a plurality of machine learned algorithm configured to generate a set of individual assessments includes selecting individual machine learned algorithms based on age information associated with the set of passive and active inputs.

14. The computer-implemented method of claim 6, wherein generating at least one processing result corresponding to the set of individual assessments includes providing a comparative assessment of the set of individual assessments relative to historical assessment information.

15. A non-transitory computer-readable medium containing computer-executable instructions that, when executed by a processor, configure the processor to perform operations including:
obtaining a set of passive and active inputs corresponding to user interactions with one or more devices;
processing at least one of the set of passive and active inputs and additional assessment according to a first machine learned algorithm configured to generate a first assessment;
processing at least one of the set of passive and active inputs and additional assessment according to a second machine learned algorithm configured to generate a second assessment, wherein the first and second assessments form a set of individual assessments; and
generating at least one processing result corresponding to the set of individual assessments.

16. The non-transitory computer-readable medium of claim 15 further comprising processing at least one of the set of passive and active inputs and additional assessment according to a third machine learned algorithm configured to generate a third assessment, wherein the first, second and third assessments form a set of individual assessments.

17. The non-transitory computer-readable medium of claim 16 further comprising processing at least one of the set of passive and active inputs and additional assessment according to a fourth machine learned algorithm configured to generate a fourth assessment, wherein the first, second, third and fourth assessments form a set of individual assessments.

18. The non-transitory computer-readable medium of claim 15, wherein the set of passive and active inputs corresponds to a plurality of devices, wherein individual devices are configured to provide at least one of an active or passive input.

19. The non-transitory computer-readable medium of claim 15 further comprising processing the first and second assessments in parallel.

20. The non-transitory computer-readable medium of claim 15, wherein at least one assessment is dependent on an assessment, the method further comprising processing the first and second assessment in an ordered manner based on dependencies.
